(19) United States
(12) Patent Application Publication
(10) Pub. No.: US 2006/0134036 A1
(43) Pub. Date: Jun. 22, 2006

(54) Antiperspirant/deodorant compositions and methods

(76) Inventors: Neil D. Scancarella, Wyckoff, NJ (US); James Joseph Ferone, Bridgewater, NJ (US); Gale McElroy Reinhart, Middletown, NJ (US); Vijay Kumar Joshi, Livingston, NJ (US); Philip Franco, Ocean Grove, NJ (US); Michael David Helman, Edison, NJ (US)

Correspondence Address: Julie Blackburn;Revlon Consumer Products Corporation, Law Department, 237 Park Avenue, New York, NY, 10017, US

(21) Appl. No.: 11/017,278

(22) Filed: Dec. 20, 2004

(51) Int. Cl. A61K 8/81; A61K 8/26

(52) U.S. Cl. 424/65

(57) ABSTRACT

An antiperspirant composition comprising at least one antiperspirant or deodorant active and at least one retentive filler, where the active is solubilized in a polar solvent and contained within the at least one retentive filler and a method for treating skin.

### TECHNICAL FIELD

The invention is in the field of antiperspirant or deodorant compositions and methods for inhibiting perspiration using such compositions.

### BACKGROUND OF THE INVENTION

Antiperspirants generally exist in the solid stick, roll-on, spray, or soft solid form. The various types of formulations may contain water, or may be anhydrous. Each form exhibits certain drawbacks. For example, while the solid stick form is convenient and easy to apply, the ingredients required to form a stick may contribute to certain undesirable properties such as stickiness or greasiness upon application. Similarly, liquid forms must be sold in certain types of components that contain the liquid formula and dispense it cleanly without leakage. When antiperspirants are in the soft solid or cream form, the product is applied either digitally, or by using certain types of containers that have dispensing domes with pores that permit the soft solid to exude when the formula is expressed from the container by turning a ratchet wheel. While each type of formula provides benefits, they are not without their accompanying drawbacks. One common problem with the stick, roll-on, and soft-solid forms is that they sometimes leave a sticky residue on the axillary area. Formulators have tried to address this issue by including various types of absorptive particulates, such as powders, in the formulas. However, the powders sometimes cause the formulas to exhibit undesirable properties such as chalkiness when in the form of a stick.

Antiperspirants in the powder form are very desirable. As there is little or no free liquid in this form, the absorptive qualities of this type of formula may be better than that found in the other forms. This aids in keeping the axilla area dry after the antiperspirant is applied. However, such issues could be resolved if the antiperspirant could be applied to the axilla in a form that is easy to work with, such as a powder, liquid, paste, or solid, and after application, forms a sheer, powdery finish on the skin.

Accordingly, it is an object of the invention to provide an antiperspirant or deodorant composition that is in the form of a powder, liquid or paste, and after application to the skin forms a sheer, powdery finish on the skin.

It is a further object of the invention to provide an antiperspirant composition containing a retentive filler into which is impregnated a solubilized antiperspirant or deodorant active, and wherein when the formula is applied to the axillary area the solubilized active is expressed from the retentive filler onto the skin and the retentive filler may serve as a collection device for perspiration and other skin secretions, and where the retentive filler is in the powder form.

It is a further object of the invention to provide an aqueous based antiperspirant or deodorant composition where the antiperspirant is solubilized in the aqueous phase and the aqueous phase is impregnated in a retentive filler.

It is a further object of the invention to provide an aqueous based antiperspirant or deodorant composition wherein the active is solubilized in an aqueous phase and the aqueous phase is impregnated into a retentive filler that is capable of releasing the solubilized active upon application of pressure such as that found when the composition is applied to the skin.

### SUMMARY OF THE INVENTION

The invention is directed to an antiperspirant composition comprising at least one antiperspirant salt solubilized in a polar solvent and at least one retentive filler, where the solubilized antiperspirant salt is impregnated into the at least one retentive filler.

The invention is also directed to a deodorant composition comprising at least one deodorant active ingredient solubilized in a polar solvent and at least one retentive filler, wherein the solubilized deodorant active ingredient is impregnated within the at least one retentive filler.

The invention is further directed to a antiperspirant or deodorant composition and method wherein the antiperspirant or deodorant active ingredient is solubilized in a polar solvent and impregnated within at least one retentive filler such that when the composition is applied to the axillary area the solubilized active is expressed, at least in part, from the retentive filler onto the skin and wherein the empty retentive filler may serve a collection function for perspiration or or other skin exudates.

The invention is further directed to an antiperspirant or deodorant composition comprising at least one antiperspirant or deodorant active solubilized in a polar solvent and impregnated within at least one retentive filler, wherein when the solubilized active is impregnated within the retentive filler the composition is in the powder form, and when the the solubilized active is expressed from the retentive filler the composition forms a liquid or paste that, when applied to the skin, provides a sheer, powdery finish.

### DETAILED DESCRIPTION

The ingredients used in the compositions of the invention will be further described herein, with all percentages mentioned being percentages by weight unless otherwise indicated.

The compositions of the invention comprise at least one deodorant or antiperspirant ingredient that is solublized in a polar solvent. The term “solubilized” when used herein means that the active ingredient is at least partially solubilized in the polar solvent. The solubilized ingredient is then contained within the at least one retentive filler that is also found in the composition.

I. The Polar Solvent

The composition of the invention contains at least one polar solvent, which may be water, or non-aqueous solvents such as ethanol, isopropanol, or glycols such as butylene glycol, propylene glycol, glycerin, and the like. The composition contains from about 0.1-95%, preferably about 0.5-80%, more preferably about 1-75% by weight of the total composition of polar solvent. The polar solvent must be capable of solubilizing, at least in part, the antiperspirant or deodorant actives that are used in the composition. Preferably the polar solvent is water, or a mixture of water and one or more of the non-aqueous polar solvents mentioned herein.

II. The Antiperspirant or Deodorant Active

The composition of the invention contains at least one antiperspirant or deodorant active solubilized in the polar solvent.

A. Antiperspirant Active

Suitable antiperspirant actives are those known for use in antiperspirants. Suggested ranges of active are from about 0.1-85%, preferably about 0.5-75%, more preferably about 1-60% by weight of the total composition. The term “antiperspirant active” or “antiperspirant salt” means any compound or composition having antiperspirant activity, preferably astringent metallic salts such as the inorganic and organic salts of aluminum, zirconium, and zinc, and mixtures thereof. Particularly preferred are the aluminum and zirconium salts such as aluminum halides, aluminum hydroxide halides, zirconyl oxide halides, zirconyl hydroxy halides, and mixtures thereof. Aluminum salts include those of the formula:
Al₂(OH)a(Cl)_{b}xH₂O
wherein a is from about 2 to 5; a+b=6; x is from about 1 to about 6; and wherein a, b, and x may have non-integer values. Zirconium salts include those of the formula:
ZrO(OH)2-a ClaxH₂O
wherein a is from about 1.5 to about 1.87; x is from about 1 to about 7; and wherein a and n may have non-integer values.

Examples of aluminum and zirconium salts include, but are not limited to, aluminum chloride, aluminum chlorohydrate, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, aluminum zirconium octachlorohdrate, aluminum zirconium octachloroydrex GLY, aluminum zirconium pentachlorohydrate, aluminum zirconium pentachlorohydrex GLY, aluminum zirconium tetrachlorohydrate, aluminum zirconium tetrachlorohydrex GLY, aluminum zirconium trichlorohydrate, aluminum zirconium trichlorohydrex GLY, and mixtures thereof.

Particularly preferred are zirconium salts in the form of complexes that also containing aluminum and glycine, in particular, aluminum zirconium tetrachlorohydrex gly. The antiperspirant salts used in the composition of the invention are solubilized in the polar solvent. While, preferably, the antiperspirant salts are completely dissolved in the polar solvent, in some cases small amounts of salts may not be dissolved, i.e. may remain in the crystalline or suspensoid form.

B. The Deodorant Active

The composition of the invention may be in the antiperspirant or deodorant form, or it is possible for the composition to contain both a mixture of antiperspirant salts and one or more deodorant ingredients. If the composition is a deodorant or combination antiperspirant or deodorant, the composition may contain one or more deodorant active ingredients. Suggested ranges include from about 0.01-40%, preferably about 0.1-35%, more preferably about 0.5-30% by weight of the total composition. Suitable deodorant actives include, but are not limited to, triclosan, sodium phenolsulfate, phenol, methylbenzethonium chloride, laurylpyridinium chloride, hexachlorophene, sodium bicarbonate, chloroxylenol, bromochlorophene, cetylpyridinium chloride, benzethonium chloride, and the like.

III. The Retentive Filler

The composition contains at least one retentive filler. The term “retentive filler” means a particulate that has channels, interstices, matrices, or is in the lamellar configuration, and which is capable of imbibing the solubilized antiperspirant or deodorant active within its free spaces, and wherein the solubilized active that is impregnated into the retentive filler can be expressed from it, either in whole or in part, upon application of pressure, such as the pressure that is applied when the composition is applied to the axilla. A variety of retentive fillers may be present, and suggested ranges are from about 0.1-95%, preferably about 1-85%, more preferably about 3-75% by weight of the total composition. Examples of retentive fillers are further described herein. The retentive fillers may have particle sizes ranging from about 0.01-1000 microns, preferably about 0.1-500 microns, more preferably from about 1-100 microns.

A. Lamellar Fillers

Lamellar fillers are suitable as retentive fillers. The term “lamellar” with respect to filler, means a particulate that is in the form of plates or sheets that may be joined in one or more places. Typically the sheets are layered on top of each other when in the dry state, but are capable of separating when exposed to polar solvents such as water. Examples of fillers that are in the lamellar form include talc, mica, titanated mica, boron nitride, bentonite, diatomaceous earth, fuller's earth, hectorite, kaolin, montmorillonite, attapulgite, or quaternized clays (such as hectorites or bentonites that are reacted with quaternary ammonium compounds, for example, quaternium-18 hectorite, quaternium-18 bentonite, and the like.

B. Crosspolymers

A wide variety of crosspolymers are also suitable, including organic polymers, silicone polymers, or copolymers of organic and silicone monomers. The term “crosspolymer” generally means a polymer containing groups that have crosslinked. The crosslinking will cause the polymer to form a matrix having inner channels or interstices that are capable of imbibing the solubilized active.

Organic crosspolymers include polymers of polymerized ethylenically unsaturated monomers where at least some of the monomers have crosslinkable groups which crosslink during or soon after polymerization of the polymer. The final polymer may be a homopolymer, copolymer, terpolymer, or graft or block copolymer, and may contain monomeric units such as acrylic acid, methacrylic acid or their simple C₁₋₃₀ akyl esters, styrene, ethylenically unsaturated monomer units such as ethylene, propylene, butylene, etc., vinyl monomers such as vinyl chloride, styrene, and so on.

In some cases, the crosspolymer contains one or more monomers which are esters of acrylic acid or methacrylic acid, including aliphatic esters of methacrylic acid like those obtained with the esterification of methacrylic acid or acrylic acid with an aliphatic alcohol of 1 to 30, preferably 1 to 20, more preferably 1 to 8 carbon atoms. If desired, the aliphatic alcohol may have one or more hydroxy, carboxy, or carboxylic acid groups. Also suitable are methacrylic acid or acrylic acid esters esterified with moieties containing alicyclic or bicyclic rings such as cyclohexyl or isobornyl, for example.

The ethylenically unsaturated monomer may be mono-, di-, tri-, or polyfunctional as regards the addition-polymerizable ethylenic bonds. A variety of ethylenically unsaturated monomers are suitable.

Examples of suitable monofunctional ethylenically unsaturated monomers include, but are not limited to, those of the formula: 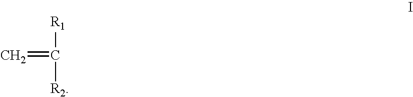
wherein R₁ is H, OH, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substitutents are C₁₋₃₀ straight or branched chain alkyl, or COOM or OCOM wherein M is H, OH, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl, carboxy, carboxylic acid, or other types of groups, or [(CH₂)ₘO]ₙH wherein m is 1-20, and n is 1-200.

More specific examples include the monofunctional ethylenically unsaturated monomer is of Formula I, above, wherein R₁ is H or a C₁₋₃₀ alkyl, and R₂ is COOM or OCOM wherein M is a C₁₋₃₀ straight or branched chain alkyl which may be substituted with one or more hydroxy groups or other types of crosslinkable groups.

Further examples include where R₁ is H or CH₃, and R₂ is COOM wherein M is a C₁₋₁₀ straight or branched chain alkyl, which may be substituted with one or more hydroxyl groups.

Di-, tri- and polyfunctional monomers, as well as oligomers, of the above monofunctional monomers may also be used to form the crosspolymer. Suitable difunctional monomers include those having the general formula: 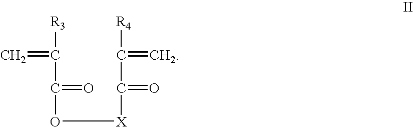
wherein R₃ and R₄ are each independently H, a C₁₋₃₀ straight or branched chain alkyl, aryl, or aralkyl; and X is [(CH₂)ₓO_{y}]_{z} wherein x is 1-20, and y is 1-20, and z is 1-100. Particularly preferred are difunctional acrylates and methacrylates, such as the compound of formula II above wherein R₃ and R₄ are CH₃ and X is [(CH₂)ₓO_{y}]_{z} wherein x is 1-4; and y is 1-6; and z is 1-10.

Trifunctional and polyfunctional monomers are also suitable for use in the polymerizable monomer to form the polymer used in the compositions of the invention. Examples of such monomers include acrylates and methacrylates such as trimethylolpropane trimethacrylate or trimethylolpropane triacrylate.

The polymers can be prepared by conventional free radical polymerization techniques in which the monomer, solvent, and polymerization initiator are charged over a 1-24 hour period of time, preferably 2-8 hours, into a conventional polymerization reactor in which the constituents are heated to about 60-175° C., preferably 80-100° C. The polymers may also be made by emulsion polymerization or suspension polymerization using conventional techniques. Also anionic polymerization or Group Transfer Polymerization (GTP) is another method by which the copolymers used in the invention may be made. GTP is well known in the art and disclosed in U.S. Pat. Nos. 4,414,372; 4,417,034; 4,508,880; 4,524,196; 4,581,428; 4,588,795; 4,598,161; 4,605,716; 4,605,716; 4,622,372; 4,656,233; 4,711,942; 4,681,918; and 4,822,859; all of which are hereby incorporated by reference.

Also suitable are polymers formed from the monomer of Formula I, above, which are cyclized, in particular, cycloalkylacrylate polymers or copolymers having the following general formulas: 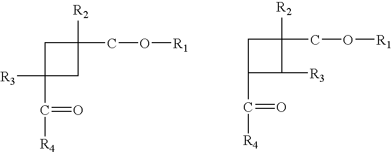
wherein R₁, R₂, R₃, and R₄ are as defined above. Typically such polymers are referred to as cycloalkylacrylate polymers.

The monomers mentioned herein can be polymerized with various types of organic groups such as propylene glycol, isocyanates, amides, etc.

One type of organic group that can be polymerized with the above monomers includes a urethane monomer. Urethanes are generally formed by the reaction of polyhydroxy compounds with diisocyanates, as follows: 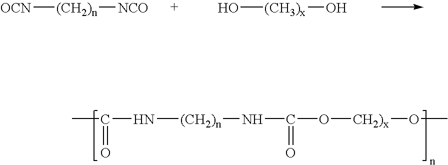
wherein x is 1-1000.

Another type of monomer that may be polymerized with the above comprise amide groups, preferably having the the following formula: 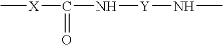
wherein X and Y are each independently linear or branched alkylene having ₁₋₄₀ carbon atoms, which may be substituted with one or more amide, hydrogen, alkyl, aryl, or halogen substituents.

Another type of organic monomer may be alpha or beta pinenes, or terpenes, abietic acid, and the like.

Suitable crosslinked retentive fillers may also be made by polymerizing ethylenically unsaturated monomers which comprise vinyl ester groups either alone or in combination with other monomers including silicon monomers, other ethylenically unsaturated monomers, or organic groups such as amides, urethanes, glycols, and the like. The various types of monomers or moieties may be incorporated into the film forming polymer by way of free radical polymerization, addition polymerization, or by formation of grafts and blocks which are attached to the growing polymer chain according to processes known in the art. Preferably the film forming polymer is an organic synthetic polymer obtained by polymerizing ethylenically unsaturated monomers comprised of vinyl ester groups and optionally organic or silicon groups or other types of ethylenically unsaturated monomers.

Other types of retentive fillers may be polymerized and crosslinked polymers having one or more vinyl ester monomers having the following general formula: 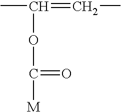
wherein M is H, or a straight or branched chain C₁₋₁₀₀ alkyl, preferably a C₁₋₅₀ alkyl, more preferably a C₁₋₄₅ alkyl which may be saturated or unsaturated, or substituted or unsubstituted, where the substituents include hydroxyl, ethoxy, amide or amine, halogen, alkyloxy, alkyloxycarbonyl, and the like. Preferably, M is H or a straight or branched chain alkyl having from 1 to 30 carbon atoms. The rentive filler may be a homopolymer or copolymer having the vinyl ester monomers either alone or in combination with other ethylenically unsaturated monomers, organic groups, or silicon monomers.

Suitable other monomers that may be copolymerized with the vinyl ester monomer include those having siloxane groups, including but not limited to those of the formula: 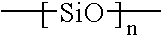
wherein n ranges from 1-1,000,000. The silicon monomers are preferably polymerized into a siloxane polymer then attached to the polymer chain by attaching a terminal organic group having olefinic unsaturation such as ethylene or propylene, to the siloxane, then reacting the unsaturated group with a suitable reactive site on the polymer to graft the siloxane chain to the polymer.

Various types of organic groups may be polymerized with the vinyl ester monomers including but not limited to urethane, amide, polyalkylene glycols, and the like as set forth above.

The vinyl ester monomers may also be copolymerized with other ethylenically unsaturated monomers that are not vinyl esters, including those set forth above.

Most preferred is where the crosspolymer is a polymer of crosslinked methacrylic acid esters or crosslinked polystyrene. One type of crosslinked methacrylic acid ester is a crosslinked polymethylmethacrylate having the INCI name methyl methacrylate crosspolymer, which may be purchased from Presperse Inc., in Piscataway, N.J., and is available under the tradename Ganzpearl. Other types of crosspolymers that are suitable include allyl methacrylates crosspolymer or HDI trimethylol hexyllactone crosspolymer, the latter being a polymer that is the cross-linked condensation polymer formed from the reaction of hexyldiisocyanate with the esterification product of trimethyloipropane with 6 to 7 moles of hexyllactone. One commercial source of HDI trimethylollactone crosspolymer is Kobo Products Inc., sold under the trade name BPD-800, which is a particulate material. On commercial sources for allyl methacrylates crosspolymer is Amcol Health & Beauty Solutions under the trade name Poly Pore E 200.

C. Silica and Silica Derivatives

Also suitable for use as the retentive fillers are various types of silicas or silicates. Examples of such silicates include those typically found in lamellar or porous form such as silica, fumed silica, calcium silicate, aluminum silicate, hydrated silica, magnesium aluminum silicate, magnesium trisilicate, silica silylate, or silicas that are substituted with hydrophobic or hydrophilic groups such as C₁₋₆ alkyl groups, C₁₋₆ alkoxy groups, and the like. Some preferred types of retentive filler that may be used in the compositions of the invention include silica, silica silylate or mixtures thereof.

D. Cellulosics

Cellulosics may also be suitable retentive fillers. Such cellulosics are polymers containing repeating cellulose units, such as starches or modified starches, either as homopolymers or copolymerized with other cellulose monomers or organic monomers. Such cellulosics may also contain alkali metal or alkaline earth metal substituents. The cellulosics may be substituted with one or more groups that confer hydrophobicity or hydrophilicity. Examples of suitable cellulosics include starch, starch substituted with C₁₋₁₀ alkyl or alkoxy groups including methyl, ethyl, propyl, methoxy, ethoxy, propoxy, etc., or starch substituted with alkali or alkaline earth metals such as sodium, potassium, magnesium, aluminum, and so on.

Also suitable are cellulosics such as starch that may be copolymerized with succinimates, succinates, or succinimides, or derivatives thereof, including materials such as aluminum starch octenylsuccinate, and the like. Particularly preferred starches are hydroxypropyl starch, hydroxyethyl starch, sodium carboxymethyl starch, aluminum starch octenylsuccinate, corn starch, rice starch, microcrystalline cellulose, maltodextrin, aluminum starch, dextran, glyceryl starch, and the like.

E. Resins

Also suitable as the retentive filler are various resins including silicone resins, organic resins, or copolymers thereof, so long as the resin exhibits at least some internal channels and is capable of imbibing the solubilized active. In the context of this invention, the term “resin” will mean a siloxane containing enough cross-linking to provide a retentive filler having internal channels. In some cases such resins may also provide substantive, film forming properties.

Typically silicone resins are at least partially crosslinked and include those referred to as T or Q resins. The term “T” generally means “trifunctional siloxy unit” and in standard silicone nomenclature a “T” unit has the general formula:
R₁SiO_{3/2}
wherein R₁ is C₁₋₃₀, preferably C₁₋₁₀, more preferably, C₁₋₄ straight or branched chain alkyl, which may be substituted with phenyl or one or more hydroxyl groups; phenyl; alkoxy (preferably C₁₋₂₂, more preferably C₁₋₆ alkyl); or hydrogen. The SiO_{3/2} designation means that the silicon atom is bonded to three oxygen atoms when the unit is copolymerized with one or more of the other units. For example when R₁ is methyl the resulting trifunctional unit is of the formula:

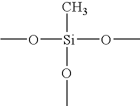

When this trifunctional unit is polymerized with one or more of the other units, the silicon atom shares three oxygen atoms with other silicon atoms, i.e. will share three halves of an oxygen atom.

The term “tetrafunctional siloxy unit” is generally designated by the letter “Q” in standard silicone nomenclature. A “Q” unit has the general formula:
SiO_{4/2}

The SiO_{4/2} designation means that the silicon shares four oxygen atoms (i.e. four halves) with other silicon atoms when the tetrafunctional unit is polymerized with one or more of the other units. The SiO_{4/2} unit is best depicted as follows:

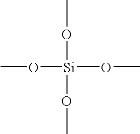

The resin may contain only T or Q units, or may be copolymerized with other siloxane units such as M or D units.

The term “monofunctional unit” or “M” means a siloxy unit that contains one silicon atom bonded to one oxygen atom, with the remaining three substituents on the silicon atom being other than oxygen. In particular, in a monofunctional siloxy unit, the oxygen atom present is shared by 2 silicon atoms when the monofunctional unit is polymerized with one or more of the other units. In silicone nomenclature used by those skilled in the art, a monofunctional siloxy unit is designated by the letter “M”, and means a unit having the general formula:
R₁R₂R₃SiO_{1/2}
wherein R₁, R₂, and R₃ are each independently C₁₋₃₀, preferably C₁₋₁₀, more preferably C₁₋₄ straight or branched chain alkyl, which may be substituted with phenyl or one or more hydroxyl groups; phenyl; alkoxy (preferably C₁₋₂₂, more preferably C₁₋₆ alkyl; or hydrogen. The SiO_{1/2} designation means that the oxygen atom in the monofunctional unit is bonded to, or shared, with another silicon atom when the monofunctional unit is polymerized with one or more of the other types of units. For example, when R₁, R₂, and R₃ are methyl the resulting monofunctional unit is of the formula:

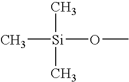

When this monofunctional unit is polymerized with one or more of the other units the oxygen atom will be shared by another silicon atom, i.e. the silicon atom in the monofunctional unit is bonded to ½ of this oxygen atom.

The term “difunctional siloxy unit” is generally designated by the letter “D” in standard silicone nomenclature. If the D unit is substituted with substituents other than methyl the “D′”designation is sometimes used, which indicates a substituent other than methyl. For purposes of this disclosure, a “D” unit has the general formula:
R₁R₂SiO_{2/2}
wherein R₁ and R₂ are defined as above. The SiO_{2/2} designation means that the silicon atom in the difunctional unit is bonded to two oxygen atoms when the unit is polymerized with one or more of the other units. For example, when R₁, R₂, are methyl the resulting difunctional unit is of the formula: 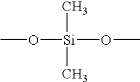
When this difunctional unit is polymerized with one or more of the other units the silicon atom will be bonded to two oxygen atoms, i.e. will share two one-halves of an oxygen atom.

The siloxane resins that form suitable retentive fillers generally comprise a majority of T or Q units, either alone or in combination with minor amounts of M or D units, the phrase “major amount” meaning that the T or Q units in the polymer are present such that the resulting polymer has sufficient porosity. The term “minor amount” means that the M or D units, if present, are not present in an amount that provides a particulate that is does not have the required degree of porosity T or MT silicones are often referred to as silsesquioxanes, and in the case where M units are present methylsilsesquioxanes. One type of T silicone that may be suitable for use as the retentive filler has units of the following general formula:
(R₁ SiO_{3/2})x
where x ranges from about 1 to 100,000, preferably about 1-50,000, more preferably about 1-10,000, and wherein R₁ is as defined above. Such MT silicones are generally referred to as polymethylsilsesquioxane which are silsesquioxanes containing methyl groups.

Examples of specific polysilsesquioxanes that may be used are manufactured by Wacker Chemie under the Resin MK designation. This polysilsesquioxane is a polymer comprise of T units and, optionally one or more D (preferably dimethylsiloxy) units. This particularly polymer may have ends capped with ethoxy groups, and/or hydroxyl groups, which may be due to how the polymers are made, e.g. condensation in aqueous or alcoholic media. Other suitable polysilsesquioxanes that may be used as the retentive filler include those manufactured by Shin-Etsu Silicones and include the “KR” series, e.g. KR-220L, 242A, and so on. These particular silicone resins may contain endcap units that are hydroxyl or alkoxy groups which may be present due to the manner in which such resins are manufactured.

Also suitable are MQ resins, which are siloxy silicate polymers having the following general formula:
[(R¹R²R³)₃SiO₁₋₂]ₓ[SiO²]_{y}
wherein R¹, R² and R³ are each independently a C₁₋₁₀ straight or branched chain alkyl or phenyl, and x and y are such that the ratio of (R¹R²R³)₃SiO_{1/2} units to SiO₂ units ranges from about 0.5 to 1 to 1.5 to 1. Preferably R¹, R² and R³ are a C₁₋₆ alkyl, and more preferably are methyl and x and y are such that the ratio of (CH₃)₃SiO_{1/2} units to SiO₂ units is about 0.75 to 1. More specifically, the trimethylsiloxysilicate thus formed contains from about 2.4 to 2.9 weight percent hydroxyl groups which is formed by the reaction of the sodium salt of silicic acid, chlorotrimethylsilane, and isopropyl alcohol. The manufacture of trimethylsiloxysilicate is set forth in U.S. Pat. Nos. 2,676,182; 3,541,205; and 3,836,437, all of which are hereby incorporated by reference. Trimethylsiloxysilicate as described is available from GE Silicones under the tradename SR-1000, which is a solid particulate material. Also suitable is Dow Corning 749 which is a mixture of volatile cyclic silicone and trimethylsiloxysilicate.

The siloxane polymeric resins that may be used as retentive fillers in the packaged composition of the invention may be made according to processes well known in the art. In general siloxane polymers are obtained by hydrolysis of silane monomers, preferably chlorosilanes. The chlorosilanes are hydrolyzed to silanols and then condensed to form siloxanes. For example, Q units are often made by hydrolyzing tetrachlorosilanes in aqueous or aqueous/alcoholic media to form the following: 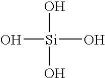
The above hydroxy substituted silane is then condensed or polymerized with other types of silanol substituted units such as: 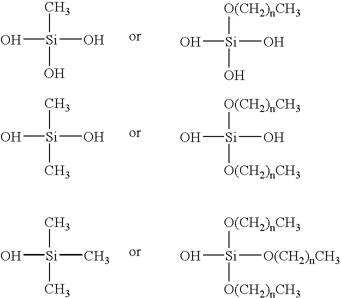
wherein n is 0-10, preferably 0-4.
Because the hydrolysis and condensation may take place in aqueous or aqueous/alcoholic media wherein the alcohols are preferably lower alkanols such as ethanol, propanol, or isopropanol, the units may have residual hydroxyl or alkoxy functionality as depicted above. Preferably, the resins are made by hydrolysis and condensation in aqueous/alcoholic media, which provides resins that have residual silanol and alkoxy functionality. In the case where the alcohol is ethanol, the result is a resin that has residual hydroxy or ethoxy functionality on the siloxane polymer. The silicone polymers that may be used in the packaged compositions of the invention are generally made in accordance with the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which is hereby incorporated by reference in its entirety.

F. Silicone Elastomers

Silicone elastomers may also be suitable retentive fillers. Silicone elastomers are generally cross-linked organosiloxane compounds prepared by reacting a dimethyl methylhydrogen siloxane with a crosslinking group comprised of a siloxane having an alkylene group having terminal olefinic unsaturation or with an organic group having an alpha or omega diene. Examples of suitable silicone elastomers for use as thixotropic agents include Dow Corning 9040, sold by Dow Corning, and various elastomeric silicones sold by Shin-Etsu under the KSG tradenames including KSG 15, KSG 16, KSG 19 and so on.

IV. Other Ingredients

The composition may contain additional ingredients that may improve the aesthetic or functional properties of the composition including, but not limited, to oils, waxes, surfactants, preservatives, inert fillers, vitamins, antioxidants, and the like. Examples of such additional ingredients include, but are not limited to, those described herein.

A. Oils

The composition may contain one or more oily ingredients. If so, suggested ranges are from about 0.01-80%, preferably from about 0.1-75%, more preferably from about 0.5-60% by weight of the total composition. Suitable oils include organic or silicone oils. The term “oil” when used herein means an ingredient that is pourable at room temperature. Such oils may be volatile or non-volatile. The term “volatile” when used herein means an oil that has a vapor pressure of greater than about 2 mm. of mercury at 20° C. The term non-volatile means an oil that has a vapor pressure of less than about 2 mm. of mercury at 20° C.

1. Volatile Oils

Volatile oils include silicones, paraffinic hydrocarbons, and the like.

(a). Volatile Silicones

Examples of suitable volatile silicones cyclic silicones are of the general formula: 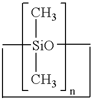
where n=3-6.

Linear volatile silicones are also suitable for use in the packaged cosmetic composition of the invention. Such silicones have the general formula:
(CH₃)₃Si—O—[Si(CH₃)₂—O]ₙ—Si(CH₃)₃
where n=0, 1, 2, 3, 4, 5, 6, or 7, preferably 0, 1, 2, 3, or 4. Such silicones include hexamethyldisiloxane, octamethyltrisiloxane, decamethyltetrasiloxane, dodecamethylpentasiloxane, and the like.

Such linear and cyclic volatile silicones are available from various commercial sources including Dow Corning Corporation and General Electric. The Dow Corning volatile silicones are sold under the tradenames Dow Corning 244, 245, 344, and 200 fluids, and have viscosities ranging from 0.5 to about 2.0 centistokes (cst) at 25° C. For example, hexamethyldisiloxane primarily comprises silicone having a viscosity of about 0.5 to 0.65 cst, while octamethyltrisiloxane primarily comprises a siloxane having a viscosity of about 1.0 cst, and decamethyltetrasiloxane comprises primarily a siloxane having a viscosity of 1.5 cst, all at 25° C.

(b). Paraffinic Hydrocarbons

Volatile paraffinic hydrocarbons that may be used in the compositions of the invention include various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 centipoise at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable. Examples of suitable volatile paraffinic hydrocarbons include isohexadecane, isododecane, or mixtures thereof.

2. Non-Volatile Oils

Examples of suitable non-volatile oils that may be used in the compositions of the invention include organic oils or silicones. Examples of such oils include those disclosed in Cosmetics, Science and Technology 27-104 (Balsam and Sagarin ed. 1972); and U.S. Pat. Nos. 4,202,879 and 5,069,897, both of which are hereby incorporated by references.

(a). Esters

Organic mono-, di-, or triesters including but not limited to those set forth herein.

(i). Monoesters

Monoesters are defined as esters formed by the reaction of a monocarboxylic acid having the formula R—COOH, wherein R is a straight or branched chain saturated or unsaturated alkyl having 2 to 150 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, ie. may have from about 6 to 30 carbon atoms. Examples of monoester oils that may be used in the compositions of the invention include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, ceyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid.

(ii). Diesters

Suitable diesters that may be used in the packaged compositions of the invention may be formed from the reaction of a dicarboxylic acid and an aliphatic or aromatic alcohol, or an aliphatic or aromatic alcohol having at least two hydroxyl groups with mono- or dicarboxylic acids. The carboxylic acids may contain from 2 to 150 carbon atoms, and may be in the straight or branched chain, saturated or unsaturated form. The carboxylic acids may be substituted with one or more hydroxyl groups. The aliphatic or aromatic alcohol may also contain 2 to 30 carbon atoms, and may be in the straight or branched chain, saturated, or unsaturated form. The aliphatic or aromatic alcohol may be substituted with one or more substituents such as hydroxyl. Preferably, one or more of the acid or alcohol is a fatty acid or alcohol, i.e. contains 14-22 carbon atoms. The carboxylic acids may also be an alpha hydroxy acid. Examples of diester oils that may be used in the compositions of the invention include diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, and so on.

(iii). Triesters

Suitable triesters comprise the reaction product of a tricarboxylic acid and an aliphatic or aromatic alcohol, or the reaction product of an aliphatic or aromatic alcohol having three or more hydroxyl groups with various mono-, di-, or tricarboxylic acids. As with the mono- and diesters mentioned above, the acid and alcohol contain 2 to 150 carbon atoms, and may be saturated or unsatured, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate, trioctyldodecyl citrate, trioctydodecyl citrate dilinoleate, tridecyl behenate, tridecyl cocoate, tridecyl isononanoate, and so on.

(b). Hydrocarbon Oils.

Also suitable are one or more hydrocarbons such as paraffins and olefins, preferably those having greater than 20 carbon atoms. Examples of such hydrocarbon oils include C₂₄₋₂₈ olefins, C₃₀₋₄₅ olefins, C₂₀₋₄₀ paraffins, hydrogenated polyisobutene, polyisobutene, mineral oil, pentahydrosqualene, squalene, squalane, and mixtures thereof.

(c). Lanolin Oil

Lanolin oil or derivatives thereof containing hydroxyl, alkyl, or acetyl groups, such as hydroxylated lanolin, isobutylated lanolin oil, acetylated lanolin, acetylated lanolin alcohol, and so on, may also be used in the compositions of the invention.

(d). Glyceryl Esters of Fatty Acids

The composition may comprise naturally occuring glyceryl esters of fatty acids, or triglycerides. Both vegetable and animal sources may be used. Examples of such oils include castor oil, lanolin oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable are synthetic or semi-synthetic glyceryl esters, e.g. fatty acid mono-, di-, and triglycerides which are natural fats or oils that have been modified, for example, acetylated castor oil, or mono-, di- or triesters of polyols such as glyceryl stearate, diglyceryl diiosostearate, polyglyceryl-4 isostearate, polyglyceryl-6 ricinoleate, glyceryl dioleate, glyceryl diisotearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(e). Fluorinated Oils

Also suitable as the oil are various fluorinated oils such as fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers like those disclosed in U.S. Pat. Nos. 5,183,589, 4,803,067, 5,183,588 all of which are hereby incorporated by reference, which are commercially available from Montefluos under the trademark Fomblin.

Fluoroguerbet esters are also suitable oils. The term “guerbet ester” means an ester which is formed by the reaction of a guerbet alcohol having the general formula: 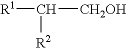
and a fluoroalcohol having the following general formula:
CF₃—(CF₂)ₙ—CH₂—CH₂—OH
wherein n is from 3 to 40.
with a carboxylic acid having the general formula:
R³COOH, or
HOOC—R³—COOH
wherein R¹, R², and R³ are each independently a straight or branched chain alkyl.

Another type of guerbet ester is a fluoro-guerbet ester, which is formed by the reaction of a guerbet alcohol and carboxylic acid (as defined above), and a fluoroalcohol having the following general formula:
CF₃—(CF₂)ₙ—CH₂—CH₂—OH
wherein n is from 3 to 40.

Examples of suitable fluoro guerbet esters are set forth in U.S. Pat. No. 5,488,121, which is hereby incorporated by reference. Suitable fluoro-guerbet esters are also set forth in U.S. Pat. No. 5,312,968 which is hereby incorporated by reference. One type of such an ester is fluorooctyldodecyl meadowfoamate, sold under the tradename Silube GME-F by Siliech, Norcross Ga.

(f). Silicones

Nonvolatile silicone oils, both water soluble and water insoluble, may also be used in the composition. Such silicones preferably have a viscosity ranging from about 10 to 600,000 centistokes, preferably 20 to 100,000 centistokes at 25° C. Suitable water insoluble silicones include amine functional silicones such as amodimethicone; phenyl substituted silicones such as bisphenylhexamethicone, phenyl trimethicone, phenyl dimethicone, or polyphenylmethylsiloxane; dimethicone, alkyl substituted dimethicones, and mixtures thereof.

Such silicones include those having the following general formula: 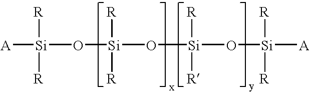
wherein R and R′ are each independently C₁₋₃₀ alkyl, phenyl or aryl, trialkylsiloxy, and x and y are each independently 0-1,000,000 with the proviso that there is at least one of either x or y, and A is siloxy endcap unit or hydroxyl. Preferred is where A is a methyl siloxy endcap unit, in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, or phenyl trimethicone. Other examples include alkyl dimethicones such as cetyl dimethicone, and the like wherein at least one R is a fatty alkyl (C₁₂, C₁₄, C₁₆, C₁₈, or C₂₂), and the other R is methyl, and A is a trimethylsiloxy endcap unit.

B. Natural or Synthetic Waxes

If desired, a variety of waxes may be used in the compositions of the invention including animal, vegetable, mineral, or silicone waxes. If present in the composition, the waxes may range from about 0.1-50%, preferably about 0.5-40%, more preferably about 1-38% by weight of the total composition. Generally such waxes have a melting point ranging from about 28 to 125° C., preferably about 30 to 100° C. Examples of animal, vegetable, or mineral waxes include acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax.

Also suitable are various types of ethylene homo- or copolymeric waxes such as polyethylene (also referred to as synthetic wax), polypropylene, and mixtures thereof. Also suitable are various types of silicone waxes, referred to as alkyl silicones, which are polymers that comprise repeating dimethylsiloxy units in combination with one or more methyl-long chain (C₁₆₋₃₀) alkyl units where the long chain alkyl is preferably a fatty chain that provides a wax-like characteristic to the silicone. Such silicones include, but are not limited to stearoxydimethicone, behenoxy dimethicone, stearyl dimethicone, cetearyl dimethicone, cetyl dimethicone, and so on. Suitable waxes are set forth in U.S. Pat. No. 5,725,845, which is hereby incorporated by reference in its entirety.

C. Surfactants

If desired, the compositions of the invention may comprise about 0.01-20%, preferably about 0.1-15%, more preferably about 0.5-10% by weight of the total composition of one or more surfactants. The surfactants present may be anionic, nonionic, cationic, zwitterionic, or amphoteric.

1. Nonionic Surfactants

(a) Organic Nonionic Surfactants

Suitable organic nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30, which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol.

Also suitable as nonionic surfactants are carboxylic acids, which are formed by the reaction of a carboxylic acid with an alkylene oxide or with a polymeric ether. The resulting products have the general formula: 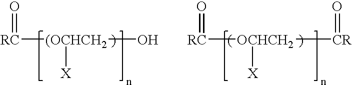
or
where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula: 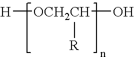
wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

(b). Silicone Surfactants

Also suitable as nonionic surfactants are various types of silicone surfactants, which are defined as silicone polymers that have at least one hydrophilic radical and at least one lipophilic radical. These silicone surfactants may be liquids or solids at room temperature. The silicone surfactant is, generally, a water-in-oil or oil-in-water type surfactant having a Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The HLB of a nonionic surfactant is the balance between the hydrophilic and lipophilic portions of the surfactant and is calculated according to the following formula:
HLB=7+11.7×log M_{w}/Mₒ
where M_{w} is the molecular weight of the hydrophilic group portion and Mₒ is the molecular weight of the lipophilic group portion.

The term “silicone surfactant” means an organosiloxane polymer containing a polymeric backbone including repeating siloxy units that may have cyclic, linear or branched repeating units, e.g. di(lower)alkylsiloxy units, preferably dimethylsiloxy units. The hydrophilic portion of the organosiloxane is generally achieved by substitution onto the polymeric backbone of a radical that confers hydrophilic properties to a portion of the molecule. The hydrophilic radical may be substituted on a terminus of the polymeric organosiloxane, or on any one or more repeating units of the polymer. In general, the repeating dimethylsiloxy units of modified polydimethylsiloxane emulsifiers are lipophilic in nature due to the methyl groups, and confer lipophilicity to the molecule. In addition, longer chain alkyl radicals, hydroxy-polypropyleneoxy radicals, or other types of lipophilic radicals may be substituted onto the siloxy backbone to confer further lipophilicity and organocompatibility. If the lipophilic portion of the molecule is due in whole or part to a specific radical, this lipophilic radical may be substituted on a terminus of the organosilicone polymer, or on any one or more repeating units of the polymer. It should also be understood that the organosiloxane polymer in accordance with the invention should have at least one hydrophilic portion and one lipophilic portion.

The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof.

The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof. The C₁₋₄₀alkyl may be non-interrupted, or interrupted by one or more oxygen atoms, a benzene ring, amides, esters, or other functional groups.

The polymeric organosiloxane surfactant used in the invention may have any of the following general formulas:
MₓQ_{y}, or
MₓT_{y}, or
MDₓD′_{y}D″_{z}M
wherein each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof. T is a trifunctional siloxy unit having the empirical formula RSiO_{1.5} or R′SiO_{1.5} wherein R is methyl and R′ is a
C₂₋₂₂ alkyl or phenyl, Q is a quadrifunctional siloxy unit having the empirical formula SiO₂, and D, D′, D″, x, y, and z are as set forth below, with the proviso that the compound contains at least one hydrophilic radical and at least one lipophilic radical. Preferred is a linear silicone of the formula:
MDₓD′_{y}D″_{z}M
wherein M=RRRSiO_{0.5}
D=RRSiO_{1.0}
D′=RR′SiO_{1.0}
D″=R′R′SiO_{1.0}

x, y, and z are each independently 0-1000,

where R is methyl or hydrogen, and R′ is a hydrophilic radical or a lipophilic radical, with the proviso that the compound contains at least one hydrophilic radical and at least one lipophilic radical.

Most preferred is wherein
M=trimethylsiloxy
D=Si[(CH₃)][(CH₂)ₙCH₃]O_{1.0} where n=0-40,
D′=Si [(CH₃)][(CH₂)ₙ—O—PE)]O_{1.0} where PE is (—C₂H₄O)ₐ(—C₃H₆O)_{b}H, o=0-40,
a=1-100 and b=1-100, and
D″=Si (CH₃)₂O_{1.0}

More specifically, suitable silicone surfactants have the formula: 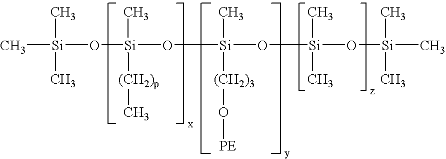
wherein p is 0-40, and
PE is (—C₂H₄O)ₐ(—C₃H₆O)_{b}—H
where x, y, z, a, and b are such that the maximum molecular weight of the polymer is approximately about 50,000.

Another type of silicone surfactant suitable for use in the compositions of the invention are emulsifiers sold by Union Carbide under the Silwet™ trademark. These surfactants are represented by the following generic formulas:
(Me₃Si)_{y-2}[(OSiMe₂)_{x/y}O-PE]_{y}
wherein PE is
—(EO)ₘ(PO)ₙR
where

R=lower alkyl or hydrogen

Me=methyl

EO is polyethyleneoxy

PO is polypropyleneoxy

m and n are each independently 1-5000

x and y are each independently 0-5000, and 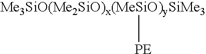
wherein PE is
—CH₂CH₂CH₂O(EO)ₘ(PO)ₙZ

where Z=lower alkyl or hydrogen, and

Me, m, n, x, y, EO and PO are as described above, with the proviso that the molecule contains a lipophilic portion and a hydrophilic portion. Again, the lipophilic portion can be supplied by a sufficient number of methyl groups on the polymer.

As with both types of silicone surfactants, the hydrophilic radical can be substituted on the terminal portions of the silicone, or in other words in the alpha or omega positions or both.

Also suitable as nonionic silicone surfactants are hydroxy-substituted silicones such as dimethiconol, which is defined as a dimethyl silicone substituted with terminal hydroxy groups.

Examples of silicone surfactants are those sold by Dow Corning under the tradename Dow Corning 3225C Formulation Aid, Dow Corning 190 Surfactant, Dow Corning 193 Surfactant, Dow Corning Q2-5200, Abil WE97, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention.

2. Anionic Surfactants

If desired the composition may contain one or more anionic surfactants. If so, suggested ranges of anionic surfactant range from about 0.01-25%, preferably 0.5-20%, more preferably about 1-15% by weight of the total composition. Suitable anionic surfactants include alkyl and alkyl ether sulfates generally having the formula ROSO₃M and RO(C₂H₄O)ₓSO₃M wherein R is alkyl or alkenyl of from about 10 to 20 carbon atoms, x is 1 to about 10 and M is a water soluble cation such as ammonium, sodium, potassium, or triethanolamine cation.

Another type of anionic surfactant which may be used in the compositions of the invention are water soluble salts of organic, sulfuric acid reaction products of the general formula:
R₁—SO₃-M
wherein R₁ is a straight or branched chain, saturated aliphatic hydrocarbon radical having from about 8 to about 24 carbon atoms, preferably 12 to about 18 carbon atoms; and M is a cation.

Examples of such anionic surfactants are salts of organic sulfuric acid reaction products of hydrocarbons such as n-paraffins having 8 to 24 carbon atoms, and a sulfonating agent, such as sulfur trioxide.

Also suitable as anionic surfactants are reaction products of fatty acids esterified with isethionic acid and neutralized with sodium hydroxide, or fatty acids reacted with alkanolamines or ammonium hydroxides. The fatty acids may be derived from coconut oil, for example. Examples of fatty acids also include lauric acid, stearic acid, oleic acid, palmitic acid, and so on.

In addition, succinates and succinimates are suitable anionic surfactants. This class includes compounds such as disodium N-octadecylsulfosuccinate; tetrasodium N-(1,2-dicarboxyethyl)-N-octadecylsulfosuccinate; and esters of sodium sulfosuccinic acid e.g. the dihexyl ester of sodium sulfosuccinic acid, the dioctyl ester of sodium sulfosuccinic acid, and the like.

Other suitable anionic surfactants include olefin sulfonates having about 12 to 24 carbon atoms. The term “olefin sulfonate” means a compound that can be produced by sulfonation of an alpha olefin by means of uncomplexed sulfur trioxide, followed by neutralization of the acid reaction mixture in conditions such that any sultones, which have been formed in the reaction are hydrolyzed to give the corresponding hydroxy-alkanesulfonates. The alpha olefin from which the olefin sulfonate is derived is a mono-olefin having about 12 to 24 carbon atoms, preferably about 14 to 16 carbon atoms.

Other classes of suitable anionic organic surfactants are the beta-alkoxy alkane sulfonates or water soluble soaps thereof, such as the salts of C₁₀₋₂₀ fatty acids, for example coconut and tallow based soaps. Preferred salts are ammonium, potassium, and sodium salts.

Still another class of anionic surfactants include N-acyl amino acid surfactants and salts thereof (alkali, alkaline earth, and ammonium salts) having the formula: 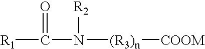
wherein R₁ is a C₈₋₂₄ alkyl or alkenyl radical, preferably C₁₀₋₁₈; R₂ is H, C₁₋₄ alkyl, phenyl, or —CH₂COOM; R₃ is CX₂— or C₁₋₂ alkoxy, wherein each X independently is H or a C₁₋₆ alkyl or alkylester, n is from 1 to 4, and M is H or a salt forming cation as described above. Examples of such surfactants are the N-acyl sarcosinates, including lauroyl sarcosinate, myristoyl sarcosinate, cocoyl sarcosinate, and oleoyl sarcosinate, preferably in sodium or potassium forms.

3. Cationic, Zwitterionic or Betaine Surfactants

Certain types of amphoteric, zwitterionic, or cationic surfactants may also be used in the compositions. Descriptions of such surfactants are set forth in U.S. Pat. No. 5,843,193, which is hereby incorporated by reference in its entirety.

Amphoteric surfactants that can be used in the compositions of the invention are generally described as derivatives of aliphatic secondary or tertiary amines wherein one aliphatic radical is a straight or branched chain alkyl of 8 to 18 carbon atoms and the other aliphatic radical contains an anionic group such as carboxy, sulfonate, sulfate, phosphate, or phosphonate.

Suitable amphoteric surfactants may be imidazolinium compounds having the general formula: 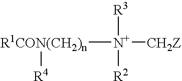
wherein R¹ is C₈₋₂₂ alkyl or alkenyl, preferably C₁₂₋₁₆; R² is hydrogen or CH₂CO₂M, R³ is CH₂CH₂OH or CH₂CH₂OCH₂CHCOOM; R⁴ is hydrogen, CH₂CH₂OH, or CH₂CH₂OCH₂CH₂COOM, Z is CO₂M or CH₂CO₂M, n is 2 or 3, preferably 2, M is hydrogen or a cation such as an alkali metal, alkaline earth metal, ammonium, or alkanol ammonium cation. Examples of such materials are marketed under the tradename MIRANOL, by Miranol, Inc.

Also, suitable amphoteric surfactants are monocarboxylates or dicarboxylates such as cocamphocarboxypropionate, cocoamphocarboxypropionic acid, cocamphocarboxyglycinate, and cocoamphoacetate.

Other types of amphoteric surfactants include aminoalkanoates of the formula
R—NH(CH₂)ₙCOOM
or iminodialkanoates of the formula:
R—N[(CH₂)ₘCOOM]₂
and mixtures thereof; wherein n and m are 1 to 4, R is C₈₋₂₂ alkyl or alkenyl, and M is hydrogen, alkali metal, alkaline earth metal, ammonium or alkanolammonium. Examples of such amphoteric surfactants include n-alkylaminopropionates and n-alkyliminodipropionates, which are sold under the trade name MIRATAINE by Miranol, Inc. or DERIPHAT by Henkel, for example N-lauryl-beta-amino propionic acid, N-lauryl-beta-imino-dipropionic acid, or mixtures thereof.

Zwitterionic surfactants are also suitable for use in the compositions of the invention. The general formula for such surfactants is: 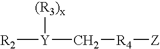
wherein R₂ contains an alkyl, alkenyl or hydroxy alkyl radical of from about 8 to about 18 carbon atoms, from 0 to about 10 ethylene oxide moieties and 0 or 1 glyceryl moiety; Y is selected from the group consisting of nitrogen, phosphorus, and sulfur atoms; R₃ is an alkyl or monohydroxyalkyl group containing about 1 to 3 carbon atoms; X is 1 when Y is a sulfur atom, and 2 when Y is a nitrogen or phosphorus atom; R₄ is an alkylene or hydroxyalkylene of from about 1 to about 4 carbon atoms, and Z is a radical selected from the group consisting of carboxylate, sulfonate, sulfate, phosphonate, and phosphate groups.

Zwitterionic surfactants include betaines, for example higher alkyl betaines such as coco dimethyl carboxymethyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxylethyl betaine, and mixtures thereof. Also suitable are sulfo- and amido-betaines such as coco dimethyl sulfopropyl betaine, stearyl dimethyl sulfopropyl betaine, and the like.

The invention will be further described in connection with the following examples, which are set forth for the purposes of illustration only.

### EXAMPLE 1

Antiperspirant formulas were prepared as follows:

| Ingredient | A | B | C |
|---|---|---|---|
| Talc and C9-15 fluoroalcohol phosphates* | 32.32 | 32.32 | 32.32 |
| HDI trimethylol hexyllactone crosspolymer, | 5.00 | 5.00 | 5.00 |
| silica** | | | |
| Butylene glycol | 5.00 | 5.00 | 5.00 |
| Water | — | 29.68 | 19.83 |
| Aluminum zirconium tetrachlorohydrate (45% | 57.68 | 28.00 | 38.00 |
| aqueous solution) | | | |

*Kobo Products, Inc. PF-5 Talc JA-46R
**BPD-800. Kobo Products, Inc.

The powders were mixed in an Osterizer blender for about 2 minutes. The blender was brushed to clean, then the blending was repeated for an additional 2 minutes. The butylenes glycol, water, and aluminum/zirconium tetrachlorohydrate solution were separately combined and mixed, then added to the powder ingredients. The liquid was added to the powder using sweep mixing with a T-blade. The compositions were mixed until uniform. Formula A provided a paste that had a draggy application when applied to the axilla. Formula B had a fluid lotion consistency and a silky application when applied to the axilla. Formula 3 was a heavier lotion that applied well and provided a silky feel on the skin.

### EXAMPLE 2

| Ingredient | A | B | C | D | E |
|---|---|---|---|---|---|
| Sodium | 1.70 | — | — | — | — |
| carboxyl | | | | | |
| methyl starch¹ | | | | | |
| Silica silylate | — | 10.00 | 10.00 | 10.00 | — |
| Talc, C9-15² | 32.12 | — | 20.00 | 20.00 | — |
| fluoroalcohol | | | | | |
| Phosphates | | | | | |
| Allyl | 3.50 | — | — | — | |
| methacrylates | | | | | |
| Crosspolymer³ | | | | | |
| HDI | — | — | — | — | 5.00 |
| trimethylol | | | | | |
| Lactone cross | | | | | |
| Polymer and | | | | | |
| silica⁴ | | | | | |
| Water | 29.68 | 62.00 | 37.00 | 22.00 | 32.00 |
| Butylene | 5.00 | — | 5.00 | 5.00 | 5.00 |
| glycol | | | | | |
| Al/Zr | 28.00 | 28.00 | 28.00 | 43.00 | 58.00 |
| tetrachloro- | | | | | |
| Hydrex gly | | | | | |
| (45% aqueous | | | | | |
| solution) | | | | | |

¹Cova Gel, LCW, a Sensient Company.
²PF Talc JA 46R, Kobo Products, Inc.
³Poly Pore E 200, Amcol Health & Beauty Solutions.
⁴BPD 800, Kobo Products, Inc.

The compositions were prepared by first combining the powder ingredients and mixing well in an Osterizer blender for about 2 minutes. The compositions were mixed with a spatula and mixed with the Osterizer blender for an additional 2 minutes. The liquid ingredients were pre-mixed, then added to the powder ingredients and mixed well. In A and E above, liquid was added to the powder using sweep agitation and a T-blade. In B, C, and D above, the liquid was added to the powder in the Osterizer and then blended. The resulting Formulas A, B, C, D provided a powder form. Formula E provided a water thin lotion in suspension. In Formula A, above, additional mixing provided a paste composition.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. An antiperspirant composition comprising at least one antiperspirant or deodorant active and at least one retentive filler, where the active is solubilized in a polar solvent and contained within the at least one retentive filler.

2. The composition of claim 1 wherein the retentive filler is a lamellar filler.

3. The composition of claim 2 wherein the lamellar filler is talc or mica.

4. The composition of claim 1 wherein the retentive filler is a crosspolymer.

5. The composition of claim 1 wherein the retentive filler is a cellulosic.

6. The composition of claim 1 wherein the retentive filler is silica or a hydrophobically modified silica.

7. The composition of claim 1 wherein the polar solvent comprises water.

8. The composition of claim 7 wherein the polar solvent comprises water and at least one mono-, di-, or polyhydric alcohol.

9. The composition of claim 4 wherein the crosspolymer is HDI trimethylollactone crosspolymer, allyl methacrylates crosspolymer, or mixtures thereof.

10. The composition of claim 5 wherein the cellulosic is starch substituted with C₁₋₁₀ alkyl or alkoxy groups, alkali or alkaline earth metals, or mixtures thereof.

11. The composition of claim 10 wherein the cellulosix is sodium carboxymethyl starch.

12. The composition of claim 1 wherein the active is one or more antiperspirant salts.

13. The composition of claim 1 wherein the active is one or more deodorant actives.

14. The composition of claim 1 wherein the active is expressed from the retentive filler when the composition is applied to the axilla.

15. A method for reducing perspiration and/or perspiration odor comprising applying to the axilla a composition comprising at least one antiperspirant or deodorant active solubilized in at least one polar solvent, and at least one retentive filler, said solubilized active being contained in at least one retentive filler and being expressed therefrom when the composition is applied to the axilla.

16. The method of claim 15 wherein the active is a deodorant active.

17. The method of claim 15 wherein the active is an antiperspirant active.

18. The method of claim 15 wherein the retentive filler is a lamellar filler, crosspolymer, cellulosic, or mixtures thereof.

19. The method of claim 18 wherein the lamellar filler is talc or mica, the crosspolymer is HDI trimethylol hexyllactone crosspolymer, or allyl methacrylates crosspolymer, and the cellulosic is sodium carboxymethyl starch.

20. The method of claim 15 wherein the polar solvent comprises water.
